(19) United States
(12) Patent Application Publication
(10) Pub. No.: US 2016/0008525 A1
(43) Pub. Date: Jan. 14, 2016

(54) Hyperoxygenation/Hyperthermia Treatment Apparatus

(71) Applicant: Jerry C Elliot, Oklahoma City, OK (US)

(72) Inventor: Jerry C Elliot, Oklahoma City, OK (US)

(21) Appl. No.: 14/329,346

(22) Filed: Jul. 11, 2014

(51) Int. Cl. A61M 1/02

(52) U.S. Cl. CPC A61M 1/0281

(57) ABSTRACT

The described invention is a hyperthermia and hyperoxygenation medical apparatus for treating diseases of the blood. The general purpose of the invention is three-fold: 1) to provide a medical tool for patient therapy and the treatment of blood viruses and disease organisms by destroying viruses and disease organisms in human and or animal blood and tissues using thermochemical treatment including heat and ozone (O3), and with the potential use of other virus and cancer fighting medications, 2) to provide an active device for hyperoxygenation and hyperthermia of human and animal blood, and 3) to provide a relatively simple treatment method for purifying stored and contaminated blood and blood components or for ensuring the condition of non-contaminated blood and blood components.

This invention optimizes and combines the advantages of proven and documented effective hyperthermia and hyperoxygenation medical techniques into a single engineering design for the research and treatment of viruses; certain types of cancers; and stored and contaminated blood supplies. It is an alternative method of specific cancer treatments and applications in diseased blood, gland and organ tissues.

### BACKGROUND OF THE INVENTION

The general field of the invention is to provide a new system that will aid 1) medical research, 2) potential therapy/treatment of viruses and disease organisms and 3) the purification of stored blood supplies and contaminated blood by the combination treatment of hyperoxygenation and/or hyperthermia as the primary treatment methods and with the ability to use medications and other pharmaceutical agents to optimize treatment.

At present, no prior art methods or devices suitable to perform the simultaneous functions of this invention are known to exist to treat and purify blood, or stored blood supplies. The difficulty of treating people infected with viruses or who have cancer is well-known. Viruses are notorious difficult to kill in the body. Likewise, cancer cells, especially those in the blood and having many of the same characteristics of the host body, mean that what kills the cancer cells may be equally likely kill the patient. Much of cancer research has focused on treating the condition without killing the patient. As a result, much of cancer treatment includes the identification of ways to kill the cancer cells or viruses that don't harm the patient “too much.”

In the past, hyperthermia and hyperoxygenation have separately been used successfully by medical practitioners. Numerous references in the medical field describe the separate use of each of these treatment methods. For various reasons, researchers have been unable to perform these treatment methodologies together on patients. This invention allows dual operation of both techniques singly or combined into one apparatus.

In addition, this invention is a new method for treating patients without dangerous radiation or chemotherapy protocols currently in use. Further, this treatment method is capable of being used in facilities other than hospitals, helping to allow providers to control costs and increase convenience and comfort to patients. Almost all treatments currently employed are in hospitals and medical facilities. With this invention, treatment may be performed in ambulatory surgical centers, doctors' offices or in a home environment.

Currently non-existent anywhere, the device is also a valuable tool to be employed in further medical research by providing the capability to combine two well know procedures, hyperthermia and hyperoxygenation, into a single delivery device to be used to sterilize human blood to increase the overall potential medical blood supply. With repeated use, the invention may be shown to have a beneficial effect on certain both organs and glands infected with harmful infectious organisms and diseases. The apparatus is much needed in medical research to determine the beneficial effects of hyperthermia and hyperoxygenation techniques and treatments, especially on certain types of cancers.

The device is a helpful research and operational tool to determine if the combination of ozone and heat improve the erythrocytic metabolism and the reactivity of the immune system with negligible cell damage.

### BRIEF SUMMARY OF THE INVENTION

It is generally known that many invasive and hostile micro-organisms prefer lower oxygen levels than the host body's cells require to remain healthy. Boosting the oxygen level revitalizes normal cells while killing viruses and other pathogens, it is also known that temperature elevation and oxygenation of blood have produced favorable disease-killing results. Ozone gas has long been recognized as a nonpolluting, purification treatment chemical for a range of applications. As an alternative disinfectant to chlorine, ozone has become more widely used as a municipal drinking water disinfectant. The invention consists of and combines two basic and well-known systems: electrical heating and ozone delivery systems combined. Treatment is not limited to the use of ozone. Throughout this specification, references to ozone should be read to include any form of molecular oxygen known in the field as beneficial to medical treatment.

The invention optimizes and combines the merits of both techniques into a single engineering design for the treatment of viruses, certain types of cancers and stored blood. At its core, the invention enables an effective method for killing viruses, cancer cells and other harmful organisms in the blood in that whereas the separate application of heat or hyperoxygenation kills, at a certain high temperature or level of hyperoxygenation, some percentage of harmful viruses or cells, in order to kill harmful viruses or cells to the extent necessary to rid the patient of disease such a high level of applied heat or hyperoxygenation must be used that the patient's life is endangered. By using the combination of hyperthermia and hyperoxygenation, cells weakened by one form of treatment may then more easily be killed by the other, owing to the lower tolerance to heat and oxygen generally displayed by harmful viruses and cells.

By the combined use of hyperoxygenation and hyperthermia, the invention takes advantage of two inherent susceptibilities of disease causing viruses and cells, allowing the application of one treatment method to weaken the subject virus or cell to the point that the other treatment method kills it. In so doing, the patient is not exposed to the more dangerous exposures of each treatment method necessary when applied individually.

After ozone and thermal exposure, different blood cells undergo different trophic, biochemical, and immunological changes that have beneficial effects on people with diseases. Ozone and thermal exposure may kill viruses in blood and accentuate the lysis of infected cells as these become poorly equipped to counteract the action of peroxides. Oxidation of components on the membrane of Peripheral Blood Mononuclear cells (lymphocytes and monocytes) may also trigger their activation with subsequent release of cytokines.

### BRIEF DESCRIPTION OF THE DRAWINGS

FIG. 1 depicts a representation of the invention showing the essential elements thereof.

FIG. 2 depicts a side view of the diffuser of the invention with ozone ports.

FIG. 3 depicts a side view of the operation of the diffuser.

### DETAILED DESCRIPTION OF THE INVENTION

The basic mode of operation in using the invention is to 1) heat the blood to a controlled temperature of approximately 105 degrees Fahrenheit (approximately 40.5 degree C.) and 2) simultaneously hyperoxygenate the blood with ozone to maximum saturation. In this combination, the high temperature kills or weakens cells and viruses in the blood susceptible to death at such temperatures. Cells and viruses which are not killed by the high temperature but which are susceptible to high oxygen levels are then killed by hyperoxygenation. Similarly, cells and viruses susceptible to high oxygen levels are killed or weakened by the hyperoxygen levels in the system. Those cells or viruses not killed are weakened sufficiently that they are then killed by the hyperthermic conditions. The blood is then cooled and returned to the body to receive additional oxygen infusion.

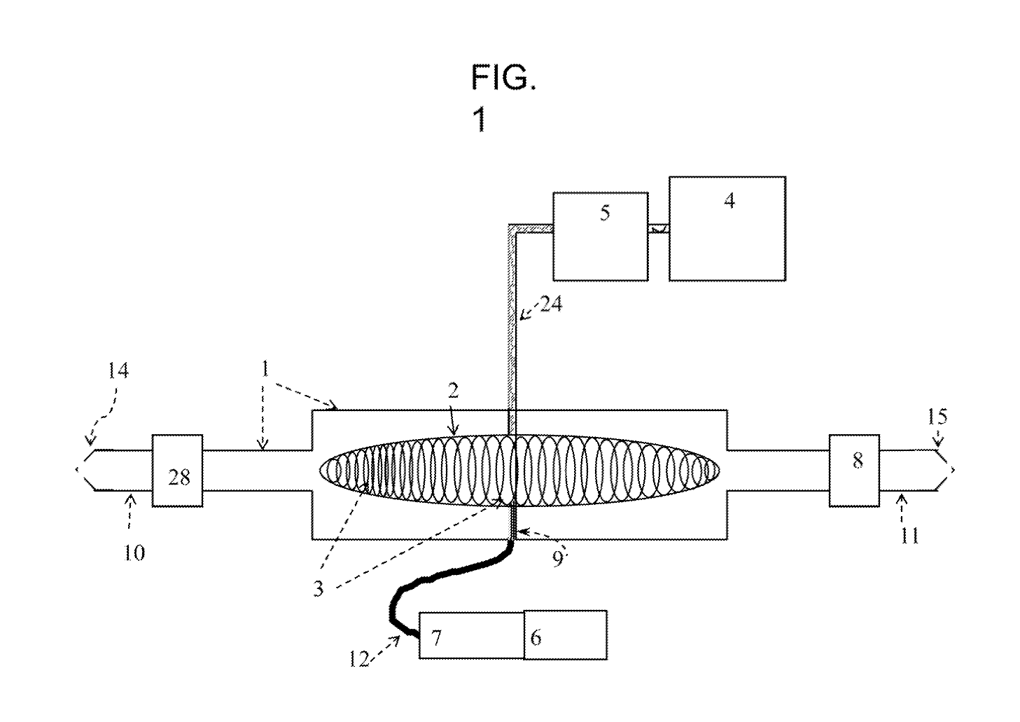

Referring now to FIG. 1, an exemplary embodiment of the invention is depicted. The patient's blood is drawn from the body through a tube inserted in a patient's vein (not depicted) to the inlet 14 of the invention 1, herein generally referred to as the chamber comprising the ozone diffuser 2 and heating element 3. Blood is drawn through any means known in the health care industry for allowing a controlled high rate of blood flow ex vivo such as might be necessary for transfusion of dialysis.

Upon flow into the chamber 1, the blood passes into a connecting tube 10. Flow into the chamber 1 is maintained by use of a pump 28. The design of the pump 28 is such that blood cells and blood components are not damaged by the pump 28 or pumping action. Upon entry into the chamber 1, blood temperature is at body temperature (98.6 degrees F.) or slightly lower, having potentially cooled while passing along the connecting tube 10. Blood flow is controlled otherwise to prevent cavitation and to ensure linear flow.

The connecting tube 10 and chamber 1 are constructed of a clear material, such as glass or other material which will not react either to heat or oxygen. The chamber 1 is a generally hollow cylinder, having a long axis substantially longer than the diameter of the cylinder and is of larger diameter than the connecting tube 10, resulting in a reduction of forward velocity of blood flow through the chamber 1. Wholly contained within the chamber 1 is an approximately helical heating element 3 disposed along the long axis of the chamber 1. The heating element 3 is disposed substantially with the ozone diffuser 2, described below. The approximately helical heating element 3 is shown as having an elliptical form to allow linear flow into and out from the chamber 1. The heating element 3 is constructed of a material which does not react either to or with the blood or diffuser 2.

The design of the chamber 1 is such that the blood flow through the chamber is sufficiently long, in terms of time, as to ensure uniform heating of the blood and all blood components along the heating coil 3 to the necessary temperature while ensuring accurate control of the temperature of the blood throughout the chamber 1. The heating coil 3 is controlled by a regulator 7 and separate power supply 6.

An ozone source is employed to contain or produce a supply of high concentration ozone, which is then dispose into an ozone supply 4 connected to the diffuser 2 via an ozone connection tube 24 for infusion of ozone into the blood. Ozone pressure in the diffuser 2 is controlled by a pressure regulator 5.

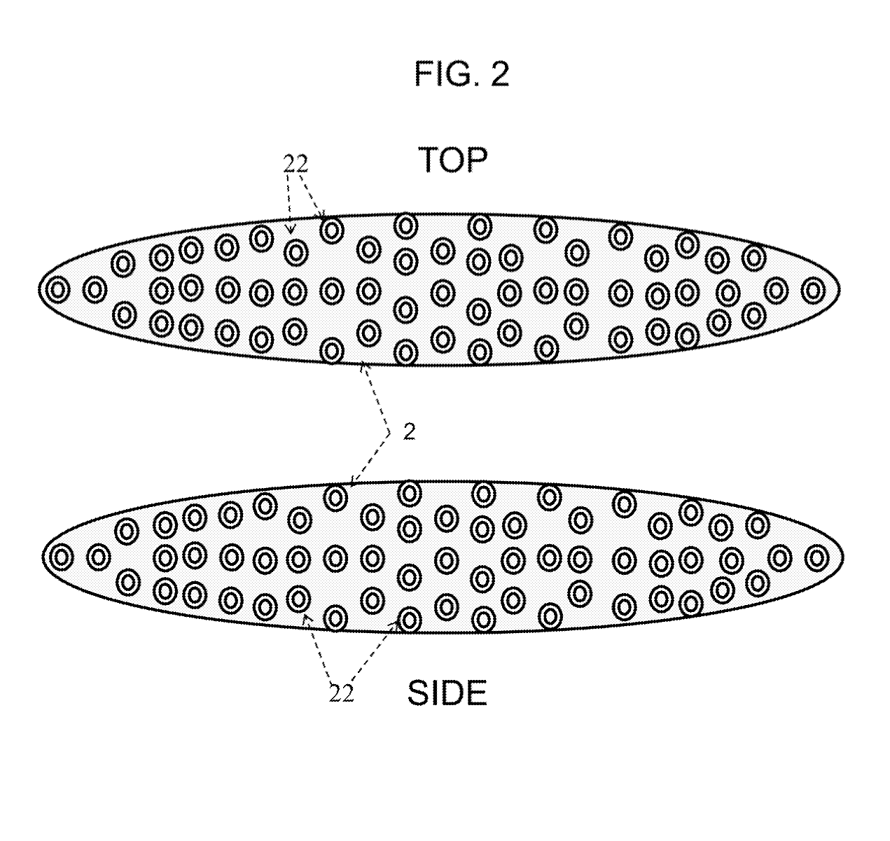

Referring now to FIG. 2, the diffuser 2 is ideally an elliptical solid in shape and hollow. The form of the diffuser 2 is such that it is disposable within the heating element 3. Upon the surface of the diffuser 2 are disposed a plurality of pores 22 along the entire surface. The number and size of pores is determined by level of ozone or oxygen saturation required or desired.

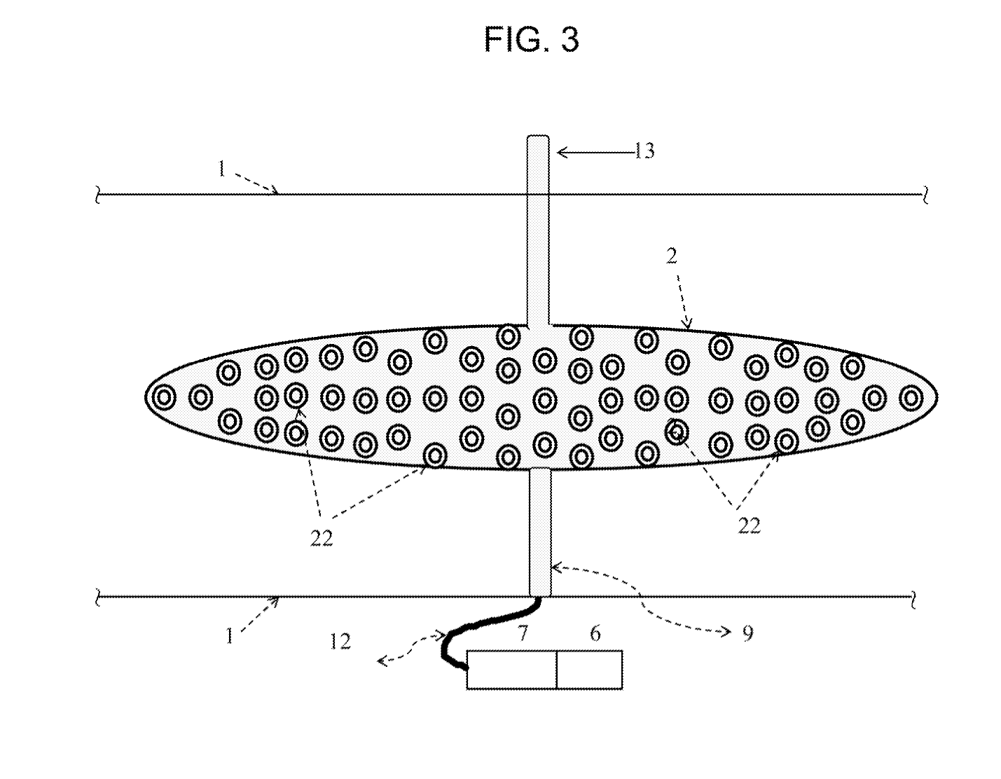

Referring now to FIG. 3, the ozone is infused into the blood via pressure differentials created by the ozone pores 22 which disperse the ozone uniformly across the blood flow through the chamber 1. Ozone not absorbed into the blood is vented from the chamber 1 via a vent 13. The diffuser 2 is mounted within the chamber 1 upon one or more support columns 9. The support columns 9 are of sufficiently small diameter so as to prevent interference with blood flow or to produce eddies or other nonlinear flow.

At least one support column 9 is hollow. This allows a power cable 12 to be run into the diffuser 2 to power the heating element 3.

Referring back to FIG. 1, the treated blood exits via the outlet variable speed pump motor 8 through the outlet connecting tube 11, having been heated and saturated with ozone. The blood then returns to the patient's circulatory system through the connecting tube 11 and via a return tube connected to nipple 15 and back into the patient's body and internal circulatory system.

In an alternate embodiment, molecule oxygen (O2) may be saturated into the blood instead of ozone.

The approach is: 1) to use a maximum ozone concentration and predetermined temperature, while improving the rheology and biochemical characteristics of erythrocytes, causing minimal damage to them, and 2) to minimize blood cell damage during ozone saturation and hyperthermia treatment. Further, the procedure is designed to expose autologous blood to ozone and high temperatures sufficient to kill or weaken disease-causing viruses, cells or pathogens, but not too high that damages blood components (106.7 deg. F. or less, for example for the maximum temperature), followed by reinfusion in patients affected by viral diseases and neoplasia. The hyperoxygenation and hyperthermia of the blood returned to the body produces additional therapeutic results in vivo.

The electrical heating system for the heating element 3 consists of a basic AC/DC power supply unit attached to the ends of a continuously embedded coil wire 3 inside and on the interior surface of the diffuser 2 in a helical fashion so as to distribute the heating uniformly over the surface of the diffuser 2. Pressure within the diffuser 2 prevents blood flow into the interior of the diffuser 2.

The power cable 12 supplying current to the heating element 3 enters the diffuser 2 ellipsoid from the power supply 6 and regulator 7 through the support tube 9 penetrating the glass wall of the chamber 1, and is attached to the heating element 3 which is continuously embedded in the interior surface of the diffuser 2 ellipsoid in a helical fashion so as to distribute heating uniformly over the surface of the Diffuser.

Heating within the chamber 1 is controlled and regulated carefully by a current regulator 7 to achieve a blood temperature of approximately 105 degrees F. and maintained within the temperature range of approximately 105° to a maximum of approximately 106.7° F. within the chamber 1.

Having heated the blood to a controlled temperature of approximately 105° F., the blood is simultaneously oxygenated with a high concentration of ozone (O3) to a specified level of concentration regulated by a regulator 5. The ozone is infused into the blood through the plurality of pores 22 on the surface of the diffuser 2 by pressure differential created within the interior of the diffuser 2, and dispersed via pressure through the plurality of pores 22.

The combination of the heating and oxygenation effects produce a method and combined effect to remove from the blood for viruses, certain types of organ cancers including but not limited to breast and prostate glands, and disease organisms due to the high oxygen rich blood level and elevated temperature.

Blood returns to the body back into the patient's circulatory system oxygenated and heated. In an alternate embodiment, the blood is cooled to body temperature prior to return to the patient.

In an alternate embodiment, the invention may be an effective means of delivering other cancer fighting agents to the patient in addition to or other than ozone and oxygen by changing the ozone supply to an optional cancer fighting agent supply system.

In an alternate embodiment, the device may be used to treat illnesses and conditions in non-human animals, with temperatures and oxygen saturation levels scaled to allow for the needs of the individual animal so treated.

The device can be made in different sizes and dimensions to whatever scale (large or small) is necessary to accomplish its intended function and purpose. In an alternate embodiment, the invention may be used to sterilize stored blood. Sterilization of blood supplies may require a larger scale device than one used for individual human or animal treatments. Consequently, the design of the device enables homogeneous heating and oxygen/ozone mixing with the possibility of fabricating the device at variable dimensions and scales.

In various alternate embodiments, blood temperature and oxygen/ozone mixing ratios can also be controlled by the adjustment of flow rates, currents, and oxygen/ozone pressures.

The invention is usable away from hospital environments and treatment centers, and so may have tremendous use and application in the home care industries as well as military battlefield environments.

What is claimed:

1. A hyperthermia/hyperoxygenation device suitable to treat cancers, viruses and other pathogens in human or animal blood and/or body organs by simultaneously or approximately simultaneously heating blood and infusing the blood with molecular oxygen to a specified temperature and oxygen concentration ex vivo.

2. The device of claim 1 in which the temperature to which the human and/or animal blood is raised is sufficient to kill most or all cancers, viruses and other pathogens in the blood, reducing the presence of same to nearly zero post treatment while resulting in no or negligible damage to the blood and blood components.

3. The device of claim 1 in which the oxygen concentration in the human and/or animal blood is raised to a sufficient concentration to kill most or all cancers, viruses and other pathogens in the human and/or animal blood, reducing the presence of same to nearly zero post treatment while resulting in no or negligible damage to the blood and blood components.

4. The device of claim 1 in which molecular oxygen infused into the blood includes O3 (ozone) or O2 (oxygen molecule).

5. The device of claim 1 in which multiple, repeated or extended applications of the treatment may be performed in a single or multiple treatments.

6. The device of claim 1 wherein an electrical circuit with an embedded helical heating coil in an diffuser is used for the controlled, regulated heating of blood.

7. The device of claim 1, wherein a molecular oxygen infusion diffuser is designed and used to provide the infusion of molecular oxygen to a highly controllable concentration.

8. The device of claim 1 in which the configuration is portable.

9. The device of claim 1 in which the device comprises a transparent glass covering the blood flow chamber permitting observation of the color, flow and effervescence bubbling of ozone or oxygen to confirm expected operation.

10. A method of treating diseases of the blood by the simultaneous and controlled heating and hyperoxygenation of blood at suitable temperatures and oxygen concentrations to kill cancer cells, viruses and other pathogen organisms in which
blood is passed ex vivo through a device with simultaneous heating and hyperoxygenation means and in which
the temperature of the heating means of the device is controllable and in which
the oxygen concentration in the treated blood is controllable and in which
following treatment, the treated blood is returned to the body.

11. The method of claim 10 in which blood may be treated multiple times in a single sitting to reduce the level of cancer cells, virus or other pathogenic organisms effectively to zero.

12. The method of claim 10 in which molecular oxygen infused into the blood includes O3 (ozone) or O2 (oxygen molecule).

13. A hyperthermia/hyperoxygenation device suitable to treat viruses and other pathogens in human or animal blood by simultaneously or approximately simultaneously heating blood and/or infusing stored blood supplies with molecular oxygen to a specified temperature and oxygen concentration ex vivo, or with the addition of heat to the blood within a controlled temperature of less than 106.5 degrees Fahrenheit.
